(19) United States
(12) Patent Application Publication
(10) Pub. No.: US 2007/0275093 A1
(43) Pub. Date: Nov. 29, 2007

(54) Methods for Treating Non-Microbial Inflammatory Skin Conditioners

(76) Inventors: Gerald E. Pierard, Angleur (BE); Valerie Vroome, Antwerpen (BE); Geert Cauwenbergh, Plainsboro, NJ (US)

Correspondence Address: LERNER, DAVID, LITTENBERG,;KRUMHOLZ & MENTLIK, 600 SOUTH AVENUE WEST, WESTFIELD, NJ, 07090, US

(21) Appl. No.: 10/576,396

(22) PCT Filed: Oct. 19, 2004
(86) PCT No.: PCT/US04/34486

§ 371 Date: May 1, 2007

(30) Foreign Application Priority Data
Oct. 20, 2003 (US) 10689015

(51) Int. Cl. A61K 31/4174; A61K 31/496; A61K 33/30; A61P 17/00; A61P 17/02; A61P 17/06; A61P 29/00

(52) U.S. Cl. 424/642; 514/254.07; 514/396; 514/399

(57) ABSTRACT

The present invention relates generally to the field of skin care products and more particularly to topical skin applications containing azole derivatives for treatment and prevention of transepidermal water loss. The present invention may be embodied in creams, lotions, oils, sprays and other typical dosage forms for direct application to the skin.

### CROSS-REFERENCE TO RELATED APPLICATIONS

This application is a continuation-in-part of U.S. patent application Ser. No. 10/689,015, filed Oct. 20, 2003, the disclosure of which is hereby incorporated herein by reference.

### BACKGROUND OF THE INVENTION

One important source of moisture in skin is transepidermal water. Transepidermal water is the moisture that migrates upward from deeper dermal tissues to the epidermis, where it hydrates (adds water to) the stratum corneum and then evaporates into the atmosphere. This process is called transepidermal water loss (TEWL). It has been shown that a variety of skin maladies or other skin conditions can lead to, or are characterized by, increased TEWL and, therefore, impaired barrier function. A method of measuring the condition of the skin, therefore, is to measure TEWL.

Skin maladies and other skin disorders related to increased TEWL and impaired barrier function have plagued mankind for centuries. They range from temporary dry skin caused by environmental conditions to serious illnesses, which can cause incapacitation and death. Included in this range are a number of conditions that can be inflammatory in nature, such as dry skin, severe dry skin, dermatitis, psoriasis, eczema, xerosis, terosis, ichthyosis, epidermolytic hyperkeratosis, infantile seborrhoeic dermatitis, atopic dermatitis, chronic dermatitis, keratoses, pruritis, cradle cap, scales, fresh stretch marks, dermatoses, burns and erythema. These specific inflammatory skin conditions are of particular relevance to the present invention in that they are not caused by microbial, bacterial or fungal, agents.

Many of these maladies and disorders can be treated and/or prevented in a number of ways including oral ingestion of drugs, intravenous injection of drugs, dietary modification, topical applications and other therapeutic methods. Of these treatment methods, the one most preferable and convenient to patients is generally a topical application.

The stratum corneum (SC) barrier function is largely dictated by extracellular lipids consisting of a mixture of ceramides, cholesterol and fatty acids together with smaller amounts of cholesterol sulphate, glucosyl ceramides and phospholipids (Yardley, (1987) Int. J. Cosmet. Sci. 9:13-19; Elias et al., (1988) J. Invest. Dermatol. 91:3-10; Rawlings et al., (1996) Arch. Dermatol. Res. 288:383-390). Skin barrier alterations exhibit profound negative effects on skin physiology. They may exacerbate some inflammatory dermatoses by inducing or boosting micro-inflammation in the irritation cascade. As stated previously, measuring transepidermal water loss (TEWL) is a good means for assessing the SC barrier function (Bashir et al., (2001) Skin Res. Technicol. 7:40-48; Zhai et al., (1998) Int. J. Dermatol. 37:386-389; Rosado and Rodrigues, (2003) Int. J. Cosmet. Sci. 25:37-44). Experimentally, tape strippings have been employed to compromise the barrier function (Id.) and the recovery rate of TEWL reflects its repair kinetics.

Measurement of TEWL, based on the estimation of the water vapor gradient in an open chamber, is being used to support claims of cosmetics including product mildness, reduction in irritative skin reactions, skin hydration, skin repair, protective effect against UV damage and others. TEWL measurement can also screen ingredients that have a beneficial effect on the barrier function and offer the possibility to monitor in vivo, on human skin, the effect of topical treatment in an objective and non-invasive way.

So-called barrier creams aim at protecting the skin from various noxious chemical effects. They may also be used as a surrogate of the natural SC function once it is weakened. These topical products tend indeed to make partial occlusion. The real level of efficacy of barrier creams is disputed. Other topical formulations and some ions can help in correcting the excess in TEWL after damaging the SC (Rawlings et al., (1996) Arch. Dermatol. Res. 288:383-390; Denda and Kumazawa, (2002) J. Invest. Dermatol. 118:65-72). Petrolatum is one of the skin barrier surrogates. Petrolatum's moisturizing characteristics have been ascribed to the slowed water loss when applied to the skin. The effect of the addition of zinc oxide (ZnO) to form a paste is unknown on this parameter. In addition, no information is available on the effect of the many pharmacological agents that can be incorporated in pastes.

Skin-protective products claiming to be barrier creams should be shields against noxious chemicals. However, the evidence in favor of their clinical usefulness is not compelling and is even contradictory (Pigatto et al., (1992) Contact Dermatitis 2226:197). Several studies have come to the conclusion that the efficacy of most of these products was questionable or even nonexistent. When evidence of efficacy is lacking, it may be due to the dearth of suitable standardized techniques for their evaluation or to the inadequate barrier properties of the preparation (Goffin et al., (1998) Dermatology 196:434-437).

Imidazoles are antimicrobial agents with effects on bacteria, fungi and protozoa. In fact, broad-spectrum antifungal products containing miconazole have been marketed in Europe to treat diaper rash. Although, these products are recommended for treatment of diaper dermatitis when Candida albicans is involved in this condition, there has been no teaching or suggestion in the art that miconazole containing products would prevent or ameliorate skin barrier alterations, or that treatment with miconazole will alleviate inflammation where microbial infection is absent.

U.S. Pat. No. 4,911,932 discloses ointment skin care compositions containing petrolatum, zinc oxide and miconazole nitrate to treat acute inflammatory skin conditions caused by microbial agents. There is no teaching or suggestion in this patent concerning the use of such skin care compositions for treatment and prevention of inflammation and TEWL for non-microbial based conditions. That is, that miconazole nitrate would have a beneficial effect beyond its known anti-microbial properties.

Similarly, compositions containing imidazoles, and miconazole in particular, have been disclosed in connection with the treatment of acne. U.S. Pat. No. 5,648,389 to Gans et al. discloses generally a combination of a dermatologically absorbable topical antimicrobial, antibiotic, antibacterial or antifungal agent; a dermatologically absorbable alpha or beta hydroxy acid, and a dermatologically absorbable zinc compound in a suitable carrier. Again, the incorporation of an imidizole is purely for its anti-microbial function.

### SUMMARY OF THE INVENTION

Applicants have discovered that skin inflammation and/or increased TEWL associated with non-microbial skin conditions can be effectively treated by the application of a topical formulation containing an imidazole, or a dermatologically acceptable salt thereof, in a dermatologically acceptable carrier. These applications are relatively and surprisingly fast acting, and may even provide overnight relief from inflammation. Without intending to be bound by any particular theory of operation, applicants believe that the imidazole provides an anti-inflammatory effect, at least in part, by decreasing transepidermal water loss.

Accordingly, a first aspect of the present invention is directed to a method for treating a non-microbial inflammatory skin condition including administering to an individual a topical formulation of at least one imidiazole or a dermatologically acceptable salt thereof and a dermatologically acceptable carrier. In another aspect of the invention, the composition further comprises a zinc compound, preferably zinc oxide.

In a preferred embodiment, the imidazole is miconazole, ketoconazole, econazole, isoconazole or a mixture thereof. More preferably, the imidazole is miconazole. As referred to herein, the term imidazole, miconazole, ketoconazole, econazole and isoconazole includes the dermatologically active salts thereof. For example, the term miconazole also refers to miconazole nitrate. In a particularly preferred embodiment, the topical formulation includes miconazole nitrate, zinc oxide, and petrolatum.

These compositions can be employed to relieve inflammation and/or TEWL associated with non-microbial skin conditions. Specifically, such conditions include dry skin, severe dry skin, dermatitis, psoriasis, eczema, xerosis, terosis, ichthyosis, epidermolytic hyperkeratosis, infantile seborrhoeic dermatitis, atopic dermatitis, chronic dermatitis, keratoses, pruritis, cradle cap, scales, fresh stretch marks, dermatoses, burns and erythema.

The file of this patent contains at least one drawing executed in color. Copies of this patent with color drawing(s) will be provided by the Patent and Trademark Office upon request and payment of the necessary fee.

### BRIEF DESCRIPTION OF THE DRAWINGS

FIG. 1 is a graph that illustrates the dynamics of skin barrier repair, as measured by reduction in TEWL, after topical application of different skin formulations. TEWL reduction in damaged epidermis (standardized skin stripping in volunteers) was used as a measure of skin barrier protection for the skin formulations. A double blind intra-individual randomized study was conducted in accordance with the declaration of Helsinki and its subsequent amendments at the University of Liege. Fifteen volunteers were enrolled. Rings on the volar aspect of the forearms delimited seven areas of 2 cm² in size. In each subject, an equal number of successive tape strippings (Tartan tape, 19 mm wide) were performed until TEWL reached 15 g/cm²/h on all test sites. Measurements were performed using a Tewameter (C+K electronic, Cologne) according to the EEMCO recommendations (Rogiers V., Skin Pharmacol Appl Skin Physiol 2001; 14: 117-128). One of the test sites was left untreated. Each of the other test sites received 1 mg of either: (1) a medicated paste made of petrolatum, 15% ZnO and 0.25% miconazole nitrate (Zimycan, Barrier Therapeutics); (2) the miconazole nitrate-free Zimycan paste (Zimybase, Barrier Therapeutics); or (3) petrolatum (ZnO-free Zimybase). Applications were performed twice daily, and TEWL measurements were taken daily 1 h after the morning treatment.

FIG. 2 is a graph that illustrates the dynamics of skin barrier repair, as measured by reduction in TEWL, after topical application of different skin formulations. FIG. 2 is essentially the same as FIG. 1 except that each of the treated test sites received 2 mg/cm² of either: (1) a medicated paste made of petrolatum, 15% ZnO and 0.25% miconazole nitrate (Zimycan, Barrier Therapeutics); (2) the miconazole nitrate-free Zimycan paste (Zimybase, Barrier Therapeutics); or (3) petrolatum (ZnO-free Zimybase).

FIG. 3 is a scattergram that illustrates the results of treatment of patients suffering from inflammation caused by acne with a miconazole nitrate/ZnO/Petrolatum topical formulation.

### DETAILED DESCRIPTION

Since the skin barrier regulates the rate of water loss from the body, the rate of transepidermal water loss is a measure of the condition of the skin barrier. When skin is damaged, its barrier function is impaired resulting in high water loss.

Skin barrier alterations exhibit profound negative effects on skin physiology. They can induce microinflammation in the inflammatory cascade. The present invention provides a method of decreasing TEWL and/or inflammation by administering a topical formulation containing at least one imidazole and a dermatologically acceptable carrier. Preferably, the formulation also contains a zinc compound, preferably, zinc oxide. While the anti-microbial properties of the imidazoles, and particularly, miconazole, are known, it has been surprisingly found that imidazoles also have anti-inflammatory properties unrelated to their role as anti-microbials. Methods of treatment are provided based on this observation for alleviating inflammation associated with a wide variety of non-microbial inflammatory skin conditions. Such conditions may include, for example, dry skin, severe dry skin, dermatitis, psoriasis, eczema, xerosis, terosis, ichthyosis, epidermolytic hyperkeratosis, infantile seborrhoeic dermatitis, atopic dermatitis, chronic dermatitis, keratoses, pruritis, cradle cap, scales, fresh stretch marks, dermatoses, burns and erythema. The term “non-microbial,” as used herein, refers to conditions that are not caused by infection by a micro-organism, such as bacteria, viruses and fungi. The term “inflammatory” or “inflammation” relates to the local response to cellular injury that is characterized by capillary dilatation, leukocyte infiltration, redness, heat, itching and pain that serves as a mechanism for the elimination of noxious agents or damaged tissue.

Methods of the present invention involve administering a topical formulation including an imidazole in a dermatologically acceptable carrier. Preferably, the topical formulation also includes a zinc compound, and may include other ingredients. These compositions provide relief from inflammation, itching and swelling, moisturize the skin and promote the healing of rashes and skin disorders.

As previously stated, the topical formulations employed in the methods of the invention include at least one imidazole. Preferred imidazoles include miconazole, ketoconazole, econazole, and isoconazole. In general, the imidazole is present in the formulation in an amount of about 0.1% to about 10.0% based on total weight of said formulation, and preferably from about 0.25% to about 2.0%. As used herein, the term “effective amount” refers to the amount of an imidazole, zinc compound or other ingredient necessary to achieve a desired result. For example, an “effective amount” of imidazole will typically be the amount of imidazole necessary to reduce inflammation and/or decreased TEWL.

The imidazole compounds of the present invention may be used as their therapeutic, dermatological, pharmaceutical, medical, and/or cosmetically acceptable salts. Thus, reference to a particular compound will, by definition, embrace the salts thereof as well. Such salts may be prepared from pharmaceutically and chemically acceptable non-toxic acids or bases including inorganic and organic acids and inorganic and organic bases. Such salts may contain, by way of example and not by way of limitation, the following ions: acetate, benzensulfonate, benzoate, camphorsulfonate, citrate, fumarate, gluconate, hydrobromide, hydrochloride, lactate, maleate, mandelate, mucate, nitrate, pamoate, phosphate, succinate, sulfate, tartate, pyruvate and the like. Such salts may also contain the following cations: aluminum, calcium, lithium, magnesium, potassium, sodium, zinc, benzathine, chloroprocaine, choline, diethanolamine, ethylenediamine, meglumine and procaine. In a preferred embodiment, the imidazole compound is miconazole nitrate.

Preferably, the topical formulation employed in the present invention also includes a zinc compound. The zinc compound may be selected from water soluble, poorly water soluble and water insoluble zinc salts, compounds and complexes, such as zinc acetate, zinc bacitracin, zinc bromide, zinc caprylate, zinc chloride, zinc citrate, zinc fluoride, zinc formate, zinc glycinate, zinc iodate, zinc lactate, zinc nitrate, zinc nitrite, zinc oleate, zinc oxalate, zinc oxide, zinc permanganate, zinc peroxide, zinc phenolsulfonate, zinc phosphate, zinc propionate, zinc pyrophosphate, zinc ricinoleate, zinc salicylate, zinc selenate, zinc silicate, zinc selenide, zinc sulfate, zinc stearate, zinc sulfide, zinc tannate, zinc tartrate, zinc valerate, zinc peptides, and zinc protein complexes. Preferably, the zinc compound is zinc oxide. The amount of zinc compound present generally ranges from about 0.25% to about 30%, preferably from about 1% to about 20%, and more preferably about 15%, based on the weight of the total composition.

The topical formulations employed in the methods of treatment of the present invention can be therapeutic, dermatological, pharmaceutical, medical, and/or cosmetic compositions depending on the particular application for which it is to be used.

In addition to the imidazole and optional zinc compound, the topical formulations of the invention include a dermatologically acceptable carrier, e.g., a substance that is capable of delivering the other components of the formulation to the skin with acceptable application or absorption of those components by the skin. The carrier will typically include a solvent to dissolve or disperse the particular imidazole compound, and, optionally one or more excipients or other vehicle ingredients. Carriers useful in accordance with the topical formulations of the present invention may include, by way of example and not by way of limitation, water, acetone, ethanol, ethylene glycol, propylene glycol, butane-1,3-diol, acrylates copolymers, isopropyl myristate, isopropyl palmitate, mineral oil, butter(s), aloe, talc, botanical oils, botanical juices, botanical extracts, botanical powders, other botanical derivatives, lanolin, urea, petroleum preparations, tar preparations, plant or animal fats, plant or animal oils, soaps, triglycerides, and keratin(s). In a preferred embodiment, the carrier is petrolatum. Carriers suitable for use in the topical formulations of the present invention include, for example, those used to form soaps, lotions, tinctures, creams, pastes, emulsions, gels/jellies, aerosols, sprays or ointments which are non-toxic and pharmaceutically, medically, dermatologically, and/or cosmetically acceptable may also be comprised within embodiments of the present invention.

Preferably, the carrier will form the remainder of the topical formulation. Generally, the carrier will be present in an amount of about 90% to about 99.9%, preferably from about 98% to about 99.75% of the total weight of the topical formulation. When a zinc compound is also included in the formulation, generally, the carrier will be present in an amount of about 60% to about 99.65%, preferably from about 78% to about 98.75% of the total weight of the topical formulation.

Additionally, moisturizers or humectants, sunscreens, fragrances, dyes, thickening agents such as paraffin, jojoba, paba, and waxes, surfactants, occlusives, hygroscopic agents, emulsifiers, emollients, lipid-free cleansers, antioxidants and lipophilic agents, may be added to the present compositions if desired.

Moisturizers or humectants are known in the art and include, for example, materials selected from the group consisting of glycerol; guanidine; glycolic acid and glycolate salts (e.g., ammonium and quaternary alkyl ammonium); lactic acid and lactate salts (e.g., ammonium and quaternary alkyl ammonium); aloe vera in any of its variety of forms (e.g., aloe vera gel); polyhydroxy alcohols such as sorbitol, glycerol, hexanetriol, propylene glycol, butylene glycol, hexylene glycol and the like; polyethylene glycols; sugars and starches including sorbitol; sugars and starch derivatives (e.g., alkoxylated glucose); hyaluronic acid; pyrrolidone carboxylic acid; lactamide monoethanolamine; acetamide monoethanolamine; and mixtures thereof.

In addition to these and other vehicles, it shall be understood that the therapeutic, dermatological, pharmaceutical, medical, and/or cosmetic topical formulations of the present invention may include other ingredients such as those that improve or eradicate itching, irritation, pain, inflammation, age spots, keratoses, wrinkles, and other blemishes or lesions of the skin. By way of example and not by way of limitation, analgesics, anesthetics, antiacne agents, antibacterial agents, anti-yeast agents, anti-fungal agents, antiviral agents, antibiotic agents, porbiotic agents, anti-protozal agents, anti-pruritic agents, antidandruff agents, anti-dermatitis agents, anti-emetics, anti-inflammatory agents, anti-hyperkeratolyic agents, anti-dry skin agents, antiperspirants, anti-psoriatic agents, anti-seborrheic agents, hair conditioners, hair treatments, hair growth agents, anti-aging agents, anti-wrinkle agents, antihistamine agents, disinfectants, skin lightning agents, depigmenting agents, vitamins and vitamin derivatives, gamma-linolenic acid (GLA), beta carotene, quercetin, asapalene, melalucas altemifolia, dimethicone, neomycin, corticosteroids, tanning agents, sulfur agents, hormones, retinoids, griseofalvin, hydroxyzine, diphenhydramine, pramoxine, lidocaine, procaine, mepivacaine, monobenzone, erythidocaine, erythromycin, tetracycline, clindamycin, meclocline, hydroquinone, minocycline, naproxen, ibuprofen, theophylline, cromolyn, alburterol, retinoic acid and its derivatives, hydrocortisone and its derivatives, mometasone, desonide, trimcinolone, predisolone, nutracort™, salicylic acid, phospholipids, calamine, allantoin, isohexadelane, ceresin, galcipotriene, dovonex™, anthralin, betamethasone valerate, betamethasone diproprionate, trimcinolone acetonide, fluocinonide, clobetasol propionate, benzoyl peroxide, crotamition, propranolon, promethanzine, vitamin A palmitate, vitamin E acetate, vitamin D and mixtures or derivatives thereof may be added to embodiments of the present invention to improve or alter their effectiveness.

Also useful are propoxylated glycerols as described in U.S. Pat. No. 4,976,953, to Orr et al., issued Dec. 11, 1990, which is incorporated by reference herein in its entirety. Suitable moisturizers are also disclosed by Loden et al. (1994), “Product Testing—Testing of Moisturizers,” in Bioengineering of the Skin: Water and the Stratum Corneum, Elsner et al., eds, CRC Press, Boca Raton, Fla. 275.

Skin protecting agents known in the art and useful in the formulations disclosed herein include sunscreens, insecticides, insect repellants, anti-acne additives, anti-wrinkle and anti-skin atrophy additives.

A wide variety of sunscreening agents are described in U.S. Pat. No. 5,087,445, to Haffey et al., issued Feb. 11, 1992; U.S. Pat. No. 5,073,372, to Tumer et al., issued Dec. 17, 1991; U.S. Pat. No. 5,073,371, to Turner et al., issued Dec. 17, 1991; and Segarin, et al., at Chapter VIII, pages 189 et seq., of Cosmetic Science and Technology, all of which are incorporated herein by reference in their entirety. Nonlimiting examples of sunscreens which are useful in the compositions of the present invention are those selected from the group consisting of 2-ethylhexyl p-methoxycinnamate, 2-ethylhexyl N,N-dimethyl-p-aminobenzoate, p-aminobenzoic acid, 2-phenylbenzimidazole-5-sulfonic acid, octocrylene, oxybenzone, homomenthyl salicylate, octyl salicylate, 4,4′-methoxy-t-butyldibenzoylmethane, 4-isopropy dibenzoylmethane, 3-benzylidene camphor, 3-(4-methylbenzylidene) camphor, anthanilates, ultrafine titanium dioxide, zinc oxide, silica and iron oxide and mixtures thereof. Still other useful sunscreens are those disclosed in U.S. Pat. No. 4,937,370, to Sabatelli, issued Jun. 26, 1990; and U.S. Pat. No. 4,999,186, to Sabatelli et al., issued Mar. 12, 1991; these two references are incorporated by reference herein in their entirety. The sunscreening agents disclosed therein have, in a single molecule, two distinct chromophore moieties, which exhibit different ultraviolet radiation absorption spectra. One of the chromophore moieties absorbs predominantly in the UVB radiation range and the other absorbs strongly in the UVA radiation range. These sunscreening agents provide higher efficacy, broader UV absorption, lower skin penetration and longer lasting efficacy relative to conventional sunscreens. Examples of these sunscreens include those selected from the group consisting of 4-N,N-(2-ethylhexyl)methylaminobenzoic acid ester of 2,4-dihydroxybenzophenone, 4-N,N-(2-ethylhexyl)-methylaminobenzoic acid ester with 4-hydroxydibenzoylmethane, 4-N,N-(2-ethylhexyl)-methylaminobenzoic acid ester of 2-hydroxy-4-(2-hydroxyethoxy)benzophenone, 4-N,N(2-ethylhexyl)-methylaminobenzoic acid ester of 4-(2-hydroxyethoxy)dibenzoylmethane, and mixtures thereof.

Nonlimiting examples of anti-wrinkle and anti-skin atrophy actives include retinoic acid and its derivatives (e.g., cis and trans); retinol, retinyl esters, salicylic acid and derivatives thereof; sulfur-containing D and L amino acids other than cysteine and their derivatives and salts, particularly the N-acetyl derivatives; alpha-hydroxy acids, e.g., glycolic acid, and lactic acid; phytic acid, lipoic acid, lysophosphatidic acid, and skin peel agents (e.g., phenol and the like).

Nonlimiting examples of insecticides, insect repellants and anti-arthropod agents include N,N-diethyl-m-toluamide, N-aryl and N-cycloalkyl neoalkonamide compounds as described in U.S. Pat. No. 5,434,190, incorporated by reference herein, terpenoids, especially terpenoid alcohols and terpenoid-esters, aldehyde and ketones of texpenes as described in U.S. Pat. No. 5,411,992, incorporated by reference herein, oils of citronella, cedar and wintergreen as described in U.S. Pat. No. 5,106,622, incorporated by reference herein, 1-nonen-3-ol, and pyrethrum/pyrethoids as described in U.S. Pat. No. 4,668,666, incorporated by reference herein.

Antibacterial agents, such as antibiotics and bactericides, and fungicides are known in the art and are useful herein as an additional component of the topical formulation. These components may be added to treat a secondary condition, or as a preventative, but are not meant for the treatment of the particular inflammatory non-microbial skin condition. Nonlimiting examples of useful antibacterial agents and fungicides include, β-lactam drugs, quinolone drugs, ciprofloxacin, norfloxacin, tetracycline, erythromycin, amikacin, 2,4,4′-trichloro-2′-hydroxy diphenyl ether, 3,4,4′-trichlorobanilide, phenoxyethanol, phenoxy propanol, phenoxyisopropanol, doxycycline, capreomycin, chlorhexidine, chloretracycline, oxytetracycline, clindamycin, ethambutol, hexamidine isethionate, metronidazole, pentamidine, gentamicin, kanamycin, lineomycin, methacycline, methenamine, minocycline, neomycin, netilmicin, paromomycin, streptomycin, tobramycin, miconazole, tetracycline hydrochloride, erythromycin, zinc erythromycin, erythromycin estolate, erythromycin stearate, amikacin sulfate, doxycycline hydrochloride, capreomycin sulfate, chlorhexidine gluconate, chlorhexidine hydrochloride, chlortetracycline, hydrochloride, oxytetracycline hydrochloride, clindamycin hydrochloride, ethambutol hydrochloride, metronidazole hydrochloride, pentamidine hydrochloride, gentamicin sulfate, kanamcyin sulfate, lineomycin hydrochloride, methacycline hydrochloride, methenamine hippurate, methenamine mendelate, minocycline hydrochloride, neomycin sulfate, netilmicin sulfate, paromomycin sulfate, streptomycin sulfate, tobramycin sulfate, miconazole hydrochloride, amanfadine hydrochloride, amanfadine sulfate, octopirox, parachlorometa xyleneol, nystatin, tolnaftate and clotrimazole.

Skin lightening agents are known in the art and may be useful in the formulations disclosed herein. Nonlimiting examples of useful skin lightening agents include glycosides of hydroxysalicylic acid and/or the glycosides of aliphatic esters of hydroxysalicylic acid as described in U.S. Pat. No. 5,700,784, incorporated by reference herein, hydroquinone, kojic acid or a derivative thereof, especially the salts or esters thereof as described in U.S. Pat. No. 5,279,834 incorporated by reference herein, 3-hydroxy-4(H)-pyran-4-one and its 3-acyl derivatives as described in U.S. Pat. No. 4,545,982 incorporated-by reference herein, and 4-hydroxy-5-methyl-3[2H]-furanone.

Artificial tanning agents and accelerators are known in the art and are useful in the formulations disclosed herein. Nonlimiting examples of useful artificial tanning agents and accelerators include dihydroxyacetone, tyrosine, tyrosine esters such as ethyl tyrosinate, and phospho-DOPA.

Anti-acne actives are known in the art and are useful in the formulations disclosed herein. Nonlimiting examples of useful anti-acne actives include the keratolytics such as salicylic acid (o-hydroxy-benzoic acid), derivatives of salicylic acid such as 5-octanoyl salicylic acid, and resorcinol; retinoids (tretinoin, isotretinoin, motretinide, adapalene, tazarotene, azelaic acid, retinal) such as retinoic acid and its derivatives (e.g., cis and trans); sulfur-containing D and L amino acids other than cysteine and their derivatives and salts, particularly their N-acetyl derivatives; lipoic acid; antibiotics and antimicrobials such as benzoyl peroxide, octopirox, tetracycline and isomers thereof, 2,4,4′-trichloro-2′-hydroxydiphenyl ether, 3,4,4′-trichlorobanilide, azelaic acid and its derivatives, erythromycin, phenoxyethanol, phenoxypropanol, phenoxisopropanol, ethyl acetate, clindamycin and melclocycline, and the anti-inflammatory agents such as ibuprofen, naproxen, hetprofen; botanical extracts such as alnus, arnica, artemisia capillaris, asiasarum root, birch, calendula, chamomile, cnidium, comfrey, fennel, galla rhois, hawthrorn, houttuynia, hypericum, jujube, kiwi, licorice, magnolia, olive, peppermint, philodendron, salvia, sasa albo-marginata; imidazoles such as ketoconazole and elubiol, and those described in Gollnick et al., ((1998) Dermatology Sebaceous Glands, Acne and Related Disorders, 196(1):119-157)), which is incorporated by reference herein; sebostats such as flavonoids; bile salts such as scymnol sulfate and its derivatives, deoxycholate, and cholate; and combinations thereof. Preferred anti-acne agents include retinol, elubiol, antibiotics, and salicylic acid, with retinol and tretinoin being most preferred. Suitable amounts of anti-acne agents include, based upon the total weight of the composition, from about 0.01 percent to about 10 percent, and preferably from about 0.04 percent to about 5 percent.

Antiviral agents are also known in the art and useful in the formulations disclosed herein. Nonlimiting examples of antiviral agents include acyclovir, vidarabine, penciclovir, trifluridine, idoxuridine, podophyllotoxin and carbenoxolone. Again, with respect to anti-microbial agents, it is noted that the methods of treatment of the present invention are directed to non-microbial inflammatory skin conditions, although it is possible that the composition may include one or more additional anti-bacterial, anti-fungal or anti-viral compounds.

The topical formulations useful in the methods of the present invention include, but are not limited to, for example, lotions, creams, ointments, sprays, aerosols, skin patches, soap, mousses, tonics, gels or the like. They may be designed to be left on the skin and not washed shortly after application. Alternatively, the topical formulations may be applied to the desired area in the form of, for example, a lotion, cream, gel, soap, shampoo or the like which is designed to be rinsed off within a given amount of time after application.

While the amount of the topical formulation to be applied will depend upon, for example, the intended usage of the final composition, i.e., therapeutic versus maintenance regimen, and sensitivity of the individual user to the composition, typically the topical formulations of the present invention should be applied to affected body parts at regular intervals, and preferably from about 5 to about 7 times per week. More preferably, the composition is applied more frequently during the initial stages of treatment, e.g., from twice daily until the desired effect is achieved, then less frequently when maintenance is desired, e.g., from about 3 to about 5 times per week.

In a preferred embodiment wherein the topical formulation is a shampoo formulation, the shampoo is applied to wet hair, and the hair is washed in accordance with known practices. More preferably, the composition remains on the hair for greater than about 0 to about 10 minutes, and preferably from about 4 to about 7 minutes before rinsing.

In one embodiment, the formulation can also include an anti-aging agent. Examples of suitable anti-aging agents include, but are not limited to inorganic sunscreens such as titanium dioxide and zinc oxide; organic sunscreens such as octyl-methyl cinnamates and derivatives thereof; retinoids; vitamins such as vitamin E, vitamin A, vitamin C, vitamin B, and derivatives thereof such as vitamin E acetate, vitamin C palmitate, and the like; antioxidants including alpha hydroxy acid such as glycolic acid, citric acid, lactic acid, malic acid, mandelic acid, ascorbic acid, alpha-hydroxybutyric acid, alpha-hydroxyisobutyric acid, alpha-hydroxyisocaproic acid, atrrolactic acid, alpha-hydroxyisovaleric acid, ethyl pyruvate, galacturonic acid, glucopehtonic acid, glucopheptono 1,4-lactone, gluconic acid, gluconolactone, glucuronic acid, glucurronolactone, glycolic acid, isopropyl pyruvate, methyl pyruvate, mucic acid, pyruvia acid, saccharic acid, saccaric acid 1,4-lactone, tartaric acid, and tartronic acid; beta hydroxy acids such as beta-hydroxybutyric acid, beta-phenyl-lactic acid, beta-phenylpyruvic acid; botanical extracts such as green tea, soy, milk thistle, algae, aloe, angelica, bitter orange, coffee, goldthread, grapefruit, hoellen, honeysuckle, Job's tears, lithospermum, mulberry, peony, puerarua, rice, safflower, and mixtures thereof. Preferred anti-aging agents include retinoids, anti-oxidants, alpha-hydroxy acids and beta-hydroxy acid, with retinol and tretinoin being most preferred.

Suitable amounts of anti-aging agents include, based upon the total weight of the composition, from about 0.01 percent to about 10 percent, and preferably from about 0.04 percent to about 5 percent.

Another aspect of Applicants' invention stems from the discovery that transdermal penetration of a given transdermally administrable chemical can be substantially improved by incorporating into a composition containing the chemical and a transdermal penetration-enhancing amount of an imidazole.

This unexpected effect is quite useful in that it allows one to improve the transdermal delivery of the chemical from the composition, thereby allowing one to achieve the same level of efficacy with a lower overall concentration of the chemical in the formulation.

The present invention provides topical formulations for the treatment of inflammation and/or decreasing TEWL that also provide transdermal penetration effect for delivery of various chemical agents into and through the skin. Such chemical agents include, but are not limited to, antihistamines, such as for example tripelennamine, triprolidine, diphenhydramine and, chlorpheniramine, all of which may be employed either as the free base or as a pharmaceutically acceptable salt.

In addition to antihistamines, other chemical agents may also have their skin penetration enhanced by the method of the present invention. Such agents include but are not limited to, the following:

Anti-bacterials; deodorants; anti-ulcer, antispasmodic and other drugs effecting the gastrointestinal tract; NSAIDS (such as for example aspirin and ibuprofen); analgesics (such as for example aspirin and ibuprofen); antipyretics, anti-inflammatories (such as for example aspirin and ibuprofen); steroids; (such as for example prednisone, prednisolone and hydrocortisone and pharmaceutically acceptable salts thereof) antifungal agents; antihypertensive agents; sympathomimetic amines (such as for example xylometazoline, phenylephrine, naphazoline and metaproterenol); central nervous system active agents; diuretics (such as for example hydrochlorothiazide); antitussives (such as for example dextromethorphan); vasodilators (such as for example nitroglycerin); anti-nauseants; and compounds for treating motion sickness.

The term “administering” or “administration” in this context refers to co-administration of an imidazole and a transdermally administrable chemical. Preferably, the imidazole and chemical will be administered simultaneously, in the same or a separate formulation. More preferably, the imidazole and transdermally administrable chemical will be provided in the same formulation. However, this is not critical, so long as the imidazole is applied within the time necessary to achieve the desired penetration enhancing effect.

Certain of the imidazoles disclosed herein as enhancing the transdermal penetration of transdermally administrable chemicals can penetrate to an extent sufficient to exert their own pharmacological effect.

Normally, the transdermally administrable chemical and the imidazole will be present in an aqueous vehicle containing an emollient and a surfactant in amounts which will be dictated by dosage considerations and the conditions of intended use, all of which are within the ability of one skilled in the art to determine and therefore will not be described in further detail here. In a preferred embodiment the topical formulations employed will contain up to about 5.0 wt. percent of transdermally administrable chemical.

While the invention has generally been described above, the details of the present invention will be better understood by recourse to the examples, which follow.

### EXAMPLE 1

Preferred topical formulations of the present invention comprise the following ingredients, which are listed according to their percentage by weight in relation to the total weight of the composition.

Skin barrier disruption coincides with an increase in TEWL. The recovery rate to normal values of TEWL following tape stripping is a good marker for assessing the skin barrier recovery. The following example was used to compare the effect of two topical pastes (petrolatum and zinc oxide, with or without miconazole nitrate), and petrolatum alone on impaired skin barrier function.

The aim of the present study was to assess the effects of paste-derived topical formulations on skin barrier repair after controlled tape strippings. Attention was paid to the amount of the products applied to the compromised skin, and to the influence if any of miconazole nitrate incorporated in the formulations.

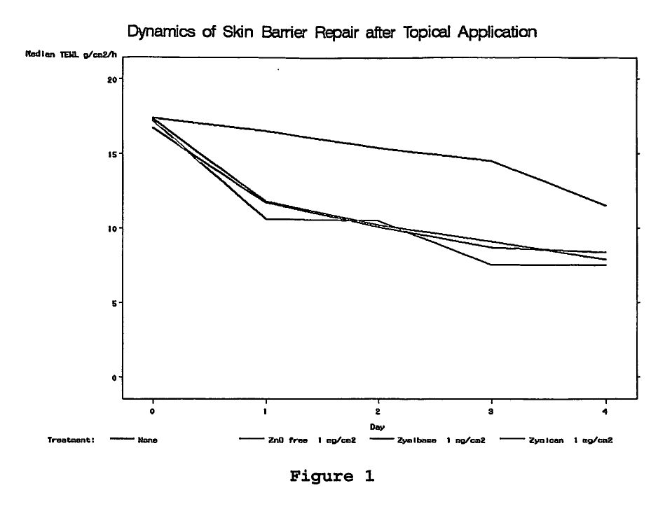

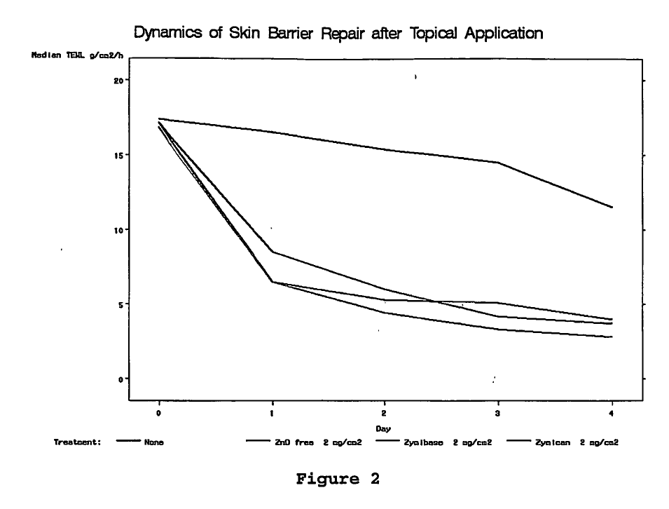

This single-blind, randomized, intra-individual study was conducted in accordance with the declaration of Helsinki and its subsequent amendments. Fifteen volunteers aged from 34 to 48 years were enrolled after they signed an informed consent. Rings on the volar aspect of the forearms delimited seven areas of 2 cm² in size. In each subject, an equal number of successive tape strippings (Tartan tape, 19 mm wide) were performed until TEWL reached 15 g/cm²/h on all test sites. One of the test sites was left untreated. Each of the other test sites received 1 of 3 formulations, which was applied twice daily for 5 days on distinct randomized test sites. The 3 formulations were (1) 1 mg or 2 mg/cm² of either a medicated paste made of petrolatum, 15% ZnO and 0.25% miconazole nitrate (Zimycan, Barrier Therapeutics); (2) the 0.25% miconazole niteate-free Zimycan paste (Zimybase, Barrier Therapeutics); or (3) petrolatum (ZnO-free Zimybase). A nurse performed applications twice daily. TEWL measurements were taken at baseline (D0) and daily for 4 days, each time 1 hour after the morning applications. The experimenter was unaware of the product randomization. A fastened skin barrier repair was induced by the 3 formulations. The effect was significantly more intense for each preparation where the largest amount had been applied. (Tables 1-4; FIGS. 1 and 2). No difference was yielded between petrolatum and the unmedicated paste. The paste containing miconazole nitrate obtained the faster recovery rate. The occlusive effect of petrolatum and a regular paste with zinc oxide helped mitigate skin barrier defect. The adjunction of miconazole nitrate improved the efficacy.

TABLE 1
TEWL-UNTREATED

| VOLUNTEER | INCLUSION | D1 | D2 | D3 | D4 |
|---|---|---|---|---|---|
| 1 | 16.3 | 17.2 | 16.1 | 14.7 | 12.2 |
| 2 | 17.7 | 15.8 | 13.4 | 11.8 | 8.7 |
| 3 | 16.8 | 16.5 | 15.2 | 15.3 | 13.8 |
| 4 | 19.2 | 21.3 | 18.5 | 16.0 | 14.1 |
| 5 | 15.9 | 14.7 | 11.0 | 8.7 | 6.5 |
| 6 | 17.4 | 17.2 | 16.2 | 15.0 | 13.1 |
| 7 | 16.2 | 16.5 | 15.4 | 15.8 | 15.3 |
| 8 | 18.6 | 17.9 | 17.1 | 16.0 | 14.1 |
| 9 | 19.2 | 18.5 | 16.9 | 14.5 | 11.5 |
| 10 | 18.0 | 16.3 | 16.1 | 13.9 | 10.2 |
| 11 | 16.8 | 13.2 | 11.4 | 9.9 | 8.6 |
| 12 | 16.2 | 15.8 | 13.9 | 12.6 | 10.0 |
| 13 | 15.7 | 11.8 | 9.6 | 7.7 | 6.1 |
| 14 | 19.1 | 20.3 | 18.9 | 16.2 | 16.3 |
| 15 | 18.3 | 16.1 | 14.9 | 11.5 | 8.8 |
| MEAN | 17.43 | 16.61 | 14.97 | 13.31 | 11.29 |
| SD | 1.25 | 2.41 | 2.69 | 2.80 | 3.17 |
| MEDIAN | 17.40 | 16.50 | 15.40 | 14.50 | 11.50 |

TABLE 2
TEWL-ZIMYBASE 1 mg/cm2

| VOLUNTEER | INCLUSION | D1 | D2 | D3 | D4 |
|---|---|---|---|---|---|
| 1 | 16.2 | 10.5 | 12.7 | 6.4 | 8.5 |
| 2 | 16.5 | 11.8 | 8.4 | 7.9 | 6.7 |
| 3 | 17.1 | 8.7 | 9.2 | 8.5 | 8.5 |
| 4 | 16.9 | 12.9 | 13.4 | 8.3 | 8.1 |
| 5 | 16.3 | 11.7 | 8.3 | 7.5 | 7.0 |
| 6 | 16.7 | 10.0 | 9.6 | 9.3 | 9.1 |
| 7 | 16.5 | 8.9 | 7.0 | 6.5 | 6.6 |
| 8 | 17.7 | 13.9 | 11.6 | 9.9 | 9.0 |
| 9 | 17.9 | 10.8 | 9.8 | 8.7 | 9.2 |
| 10 | 16.7 | 13.2 | 11.7 | 9.8 | 9.1 |
| 11 | 16.5 | 11.8 | 9.5 | 9.3 | 8.6 |
| 12 | 16.7 | 13.3 | 10.1 | 9.7 | 6.6 |
| 13 | 16.4 | 11.0 | 10.5 | 10.3 | 8.4 |
| 14 | 17.5 | 13.9 | 10.7 | 9.8 | 6.1 |
| 15 | 18.0 | 11.4 | 11.8 | 8.3 | 6.9 |
| MEAN | 16.91 | 11.59 | 10.29 | 8.68 | 7.89 |
| SD | 0.59 | 1.65 | 1.74 | 1.22 | 1.11 |
| MEDIAN | 16.70 | 11.70 | 10.10 | 8.70 | 8.40 |

TABLE 3
TEWL-ZIMYCAN 1 mg/cm2

| VOLUNTEER | INCLUSION | D1 | D2 | D3 | D4 |
|---|---|---|---|---|---|
| 1 | 16.8 | 11.7 | 11.5 | 9.9 | 7.9 |
| 2 | 17.4 | 10.6 | 8.2 | 7.7 | 6.1 |
| 3 | 16.3 | 12.9 | 10.6 | 9.3 | 8.0 |
| 4 | 18.2 | 15.5 | 14.2 | 14.1 | 10.2 |
| 5 | 16.7 | 10.2 | 8.4 | 6.1 | 4.4 |
| 6 | 16.3 | 8.3 | 12.5 | 8.1 | 7.5 |
| 7 | 16.6 | 9.8 | 6.6 | 3.2 | 3.8 |
| 8 | 18.7 | 10.4 | 8.2 | 7.6 | 7.7 |
| 9 | 18.5 | 14.3 | 11.3 | 9.8 | 8.4 |
| 10 | 17.2 | 8.5 | 9.2 | 6.4 | 8.3 |
| 11 | 17.4 | 11.4 | 7.7 | 7.5 | 7.9 |
| 12 | 16.0 | 14.2 | 10.5 | 6.3 | 4.0 |
| 13 | 15.5 | 8.6 | 11.7 | 2.8 | 6.7 |
| 14 | 17.6 | 14.3 | 11.9 | 6.7 | 3.8 |
| 15 | 17.9 | 9.9 | 6.1 | 3.5 | 3.2 |
| MEAN | 17.14 | 11.37 | 9.91 | 7.27 | 6.53 |
| SD | 0.94 | 2.35 | 2.34 | 2.92 | 2.16 |
| MEDIAN | 17.20 | 10.60 | 10.50 | 7.50 | 7.50 |

TABLE 4
TEWL-ZIMYCAN 2 mg/cm2

| VOLUNTEER | INCLUSION | D1 | D2 | D3 | D4 |
|---|---|---|---|---|---|
| 1 | 16.6 | 6.3 | 6.6 | 1.4 | 4.9 |
| 2 | 17.0 | 5.2 | 4.7 | 4.9 | 4.5 |
| 3 | 16.6 | 8.5 | 3.2 | 2.8 | 2.8 |
| 4 | 17.9 | 7.0 | 8.7 | 3.3 | 1.0 |
| 5 | 16.5 | 4.4 | 2.9 | 2.6 | 2.3 |
| 6 | 17.0 | 8.3 | 5.0 | 3.3 | 3.7 |
| 7 | 16.0 | 6.3 | 4.1 | 4.0 | 3.0 |
| 8 | 18.2 | 9.4 | 1.3 | 2.4 | 2.1 |
| 9 | 18.3 | 10.1 | 4.7 | 6.3 | 4.9 |
| 10 | 17.9 | 9.0 | 2.8 | 4.0 | 1.1 |
| 11 | 16.8 | 5.1 | 3.6 | 4.2 | 3.3 |
| 12 | 16.3 | 4.5 | 2.4 | 2.1 | 2.8 |
| 13 | 15.3 | 6.3 | 8.4 | 5.4 | 5.5 |
| 14 | 18.2 | 7.2 | 4.4 | 7.0 | 1.9 |
| 15 | 16.3 | 6.5 | 6.0 | 2.2 | 2.0 |
| MEAN | 16.99 | 6.94 | 4.59 | 3.73 | 3.05 |
| SD | 0.91 | 1.79 | 2.11 | 1.62 | 1.40 |
| MEDIAN | 16.80 | 6.50 | 4.40 | 3.30 | 2.80 |

The TEWL evolution (means for 15 human subjects) for the 7 test sites is given in Tables 1-4. Results at baseline for the different treatment sites were summarized as median values. Mixed model analysis of variance of the data obtained at day 1-4 with measurements made at the inclusion day (0) as covariate, treatment day as factor and a compound symmetry structure for the covariance matrix, indicated a significant lower TEWL for Zymican at 2 mg/cm² than for both Zymibase and ZnO-free Zymibase (Dunnett-Hsu test, two-sided versus Zymibase p=0.011, versus ZnO-free Zymibase p=0.017). No statistically significant difference could be detected between Zymibase and its ZnO-free version (unadjusted p=0.836). Using the two-sided Dunnett-Hsu test, TEWL in the miconazole group was significantly lower than that for the petrolatum+ZnO group (p=0.011) and than that for Petrolatum alone (p=0.017). Computations were carried out using the SAS 8.0.2 system for statistical analysis.

The dynamics of skin barrier repair, as measured by reduction in TEWL, after topical application of the 3 different skin formulations is illustrated in FIGS. 1 and 2. The additive effects of miconazole were unexpected. Indeed, already in the 1 mg/cm² group, there was a trend towards a significantly improved TEWL in the Petrolatum+ZnO+miconazole group versus the 2 other treatment groups. This difference in favor of the miconazole-containing formulation became significant in the groups treated with 2 mg/cm.

In order to have smooth, hydrated and non-scaly skin, an intact SC barrier function is essential. The assessment of TEWL by evaporimetry or by the passive sustainable hydration test has been shown to be a suitable tool to quantify any impairment of the barrier function (Abrams et al., (1993) J. Invest. Dermatol. 101:609-613; Van Cromphaut et al., (1999) J. Environ. Med. 1:47-50.). This function resides in the SC, which is composed of protein-rich nonviable cells and intercellular lipid domains originating from the keratinosomes. When tape stripping or treatment with an organic solvent or detergent damages the SC barrier function, a series of homeostatic processes in barrier function is immediately accelerated, and the barrier recovers to its original level. This process includes lipid synthesis, lipid processing, and the acceleration of exocytosis of lipid-containing lamellar bodies in the upper epidermis.

In comparison with untreated skin, an improved skin barrier repair was observed after applications of petrolatum and of both the medicated and unmedicated pastes. Furthermore, the miconazole nitrate paste had a somewhat greater protective effect than its unmedicated vehicle. The mechanism of action of miconazole nitrate on this aspect of skin biology is uncertain.

The present invention provides that the 3 tested formulations decreased TEWL and thus helped in repairing an impaired skin barrier function. The presence of zinc oxide did not appear to influence this effect.

Based on general knowledge it was expected that petrolatum alone would reduce TEWL in this test model. Adding ZnO to the petrolatum base did not substantially alter/improve the TEWL reduction in the volunteers. It was surprising however, that adding 0.25% miconazole to the petrolatum+ZnO base caused a significant enhancement in TEWL reduction as compared to petrolatum alone and petrolatum+ZnO.

This TEWL reducing effect of miconazole has not been described before, and suggests that certain members of the imidazole family (miconazole, ketoconazole, econazole, isoconazole and others) have, in addition to their antifungal properties, also skin barrier enhancing effects.

The data disclosed herein show that imidazoles like miconazole have skin barrier protective effects on top of the protective effects of conventional barrier creams. It is believed that, in addition to the antimicrobial effects of the imidazole moiety, this chemical group also affects the restoration of the barrier protective properties of the epidermis. Other imidazoles for topical use, for example, imidazoles such as ketoconazole, econazole and isoconazole, have similar effects to miconazaole. This could be important for the development of new topical medications for the treatment of erosive skin lesions, burns, chronic wounds and wet inflammatory skin conditions such as flexural atopic dermatitis.

As expected, petrolatum reduced TEWL in a dose dependent manner. Surprisingly, adding ZnO to the petrolatum did not cause a further improvement in TEWL, despite the increase in skin adhesivity that was observed in the treated volunteers. Finally, adding miconazole in a relatively small concentration (0.25%) showed a surprising enhancement of the TEWL reducing effects of petrolatum.

Imidazoles such as miconazole may constitute a novel class of chemicals for treatment of an impaired skin barrier function.

### EXAMPLE 2

The topical compositions of the present invention have been shown to be surprisingly fast acting in alleviating inflammation. To demonstrate this effect, a topical application was prepared containing 0.25% miconazole nitrate formulated in a zinc oxide/petrolatum base, and employed to treat patients suffering from acne. Because of the rapidity of activity, this anti-inflammatory activity was not attributable to the anti-fungal or anti-bacterial activity of the imidazole compound.

Fifteen adolescents between 15 and 17 years of age were enrolled in the study. They suffered from inflammatory papular acne of the face that remained untreated for a period of at least two months. A thin film of the medicated paste was applied to papules in the evening. During the preceding afternoon, and the next morning, erythema of the papules was measured using narrow band reflectance spectroscopy. A total of 117 lesions were measured in the 15 patients. The mean spectroscopic value (erythema score) of non-lesional, non-inflamed skin in these patients was 413.

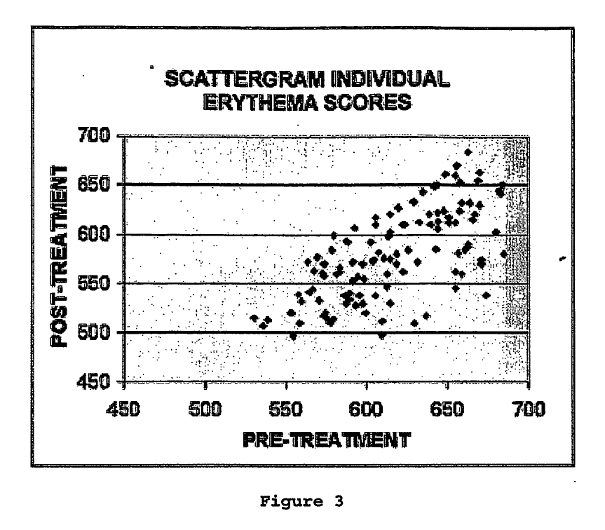

On average, the mean erythema score dropped overnight from 617 to 579; a difference of 38 units or 19% when these values are corrected against the erythema score of non-lesional facial skin (413). Comparisons were made using the 2-tailed paired t test of Student. Before the measurement was made the next morning, residual ointment was removed from the skin, to reduce the risk of interference of the vehicle with the measurements. The data obtained are shown in Table 5 and in the scattergram in FIG. 3.

TABLE 5
DATA POINTS OF SCATTERGRAM (FIG. 3)

| SUBJECT | PRE-TREATMENT | POST-TREATMENT |
|---|---|---|
| 1 | 574 | 517 |
| 2 | 610 | 498 |
| 3 | 630 | 510 |
| 4 | 592 | 572 |
| 5 | 619 | 570 |
| 6 | 593 | 607 |
| 7 | 614 | 598 |
| 8 | 635 | 643 |
| 9 | 640 | 610 |
| 10 | 643 | 585 |
| 11 | 658 | 624 |
| 12 | 555 | 497 |
| 13 | 577 | 513 |
| 14 | 594 | 528 |
| 15 | 600 | 520 |
| 16 | 603 | 592 |
| 17 | 606 | 610 |
| 18 | 682 | 644 |
| 19 | 580 | 599 |
| 20 | 584 | 566 |
| 21 | 592 | 553 |
| 22 | 598 | 570 |
| 23 | 664 | 632 |
| 24 | 615 | 530 |
| 25 | 632 | 572 |
| 26 | 655 | 545 |
| 27 | 659 | 560 |
| 28 | 564 | 572 |
| 29 | 571 | 533 |
| 30 | 588 | 593 |
| 31 | 606 | 617 |
| 32 | 624 | 610 |
| 33 | 580 | 514 |
| 34 | 587 | 538 |
| 35 | 615 | 602 |
| 36 | 620 | 627 |
| 37 | 626 | 584 |
| 38 | 637 | 517 |
| 39 | 670 | 663 |
| 40 | 684 | 650 |
| 41 | 642 | 649 |
| 42 | 663 | 684 |
| 43 | 559 | 510 |
| 44 | 590 | 534 |
| 45 | 611 | 576 |
| 46 | 619 | 580 |
| 47 | 623 | 562 |
| 48 | 669 | 655 |
| 49 | 683 | 641 |
| 50 | 575 | 520 |
| 51 | 596 | 538 |
| 52 | 613 | 547 |
| 53 | 644 | 622 |
| 54 | 655 | 660 |
| 55 | 659 | 633 |
| 56 | 666 | 615 |
| 57 | 680 | 602 |
| 58 | 578 | 510 |
| 59 | 644 | 613 |
| 60 | 655 | 562 |
| 61 | 670 | 630 |
| 62 | 588 | 530 |
| 63 | 605 | 574 |
| 64 | 610 | 512 |
| 65 | 613 | 560 |
| 66 | 629 | 633 |
| 67 | 655 | 612 |
| 68 | 657 | 581 |
| 69 | 671 | 570 |
| 70 | 553 | 520 |
| 71 | 574 | 558 |
| 72 | 579 | 513 |
| 73 | 589 | 592 |
| 74 | 590 | 540 |
| 75 | 595 | 557 |
| 76 | 606 | 538 |
| 77 | 623 | 610 |
| 78 | 644 | 650 |
| 79 | 649 | 661 |
| 80 | 651 | 617 |
| 81 | 661 | 584 |
| 82 | 663 | 590 |
| 83 | 685 | 580 |
| 84 | 565 | 541 |
| 85 | 568 | 563 |
| 86 | 570 | 577 |
| 87 | 573 | 561 |
| 88 | 574 | 570 |
| 89 | 579 | 584 |
| 90 | 584 | 562 |
| 91 | 598 | 530 |
| 92 | 615 | 620 |
| 93 | 633 | 612 |
| 94 | 648 | 624 |
| 95 | 658 | 653 |
| 96 | 530 | 515 |
| 97 | 536 | 507 |
| 98 | 539 | 513 |
| 99 | 554 | 520 |
| 100 | 558 | 539 |
| 101 | 560 | 532 |
| 102 | 567 | 544 |
| 103 | 582 | 560 |
| 104 | 599 | 555 |
| 105 | 604 | 573 |
| 106 | 608 | 582 |
| 107 | 615 | 575 |
| 108 | 630 | 633 |
| 109 | 639 | 620 |
| 110 | 644 | 606 |
| 111 | 651 | 612 |
| 112 | 656 | 670 |
| 113 | 659 | 632 |
| 114 | 663 | 588 |
| 115 | 667 | 620 |
| 116 | 671 | 574 |
| 117 | 674 | 538 |

The mean of the paired differences was 38 with the 95% confidence interval of the difference between 32 and 44. The differences between the before and after values is highly significant (p<0.0001).

It is concluded that a single overnight application of 0.25% miconazole in a ZnO/petrolatum base had a significant effect on erythema in inflamed acne lesions in the face. In light of additional tests showing an anti-inflammatory effect of 0.25% miconazole in volunteers with surfactant induced irritant dermatitis (Example 3), one can conclude that the overnight effect on inflammation in this study of acne patients was also caused by anti-inflammatory effects of miconazole.

### EXAMPLE 3

Twenty-five healthy volunteers were subjected to induced inflammation by tape stripping of skin. A topical formulation made of petrolatum, 15% ZnO and 0.25% miconazole nitrate (Zimycan, Barrier Therapeutics) was applied overnight and measurements taken thereafter. As a control, a formulation of zinc oxide and petrolatum was applied to similar tape stripping with the same objects. The results of this example are shown in Table 6, in which measurements are in relation to a base white. In Table 7, these results were adjusted for normal skin tone, or 410 (taken from 30 observations). For example taking the Zimycan Before data for subject 1 of Table 6, 533, and subtracting 410 yields 123 as shown in Table 7.

TABLE 6
Skin Stripping Study, Zimycan v. Vehicle Control

| | Zimycan | Zimycan | Vehicle | Vehicle |
|---|---|---|---|---|
| | Before | After | Before | After |
| 1 | 533 | 472 | 534 | 518 |
| 2 | 596 | 544 | 606 | 597 |
| 3 | 569 | 534 | 576 | 576 |
| 4 | 576 | 553 | 601 | 596 |
| 5 | 588 | 563 | 561 | 560 |
| 6 | 644 | 577 | 617 | 609 |
| 7 | 628 | 613 | 639 | 644 |
| 8 | 563 | 543 | 573 | 575 |
| 9 | 613 | 606 | 642 | 641 |
| 10 | 585 | 582 | 596 | 595 |
| 11 | 567 | 542 | 559 | 557 |
| 12 | 683 | 643 | 686 | 682 |
| 13 | 606 | 592 | 611 | 611 |
| 14 | 655 | 612 | 658 | 657 |
| 15 | 632 | 608 | 611 | 614 |
| 16 | 575 | 549 | 557 | 543 |
| 17 | 529 | 521 | 554 | 543 |
| 18 | 645 | 597 | 645 | 625 |
| 19 | 549 | 542 | 545 | 537 |
| 20 | 625 | 594 | 642 | 638 |
| 21 | 619 | 609 | 658 | 666 |
| 22 | 674 | 655 | 682 | 673 |
| 23 | 611 | 599 | 605 | 609 |
| 24 | 598 | 561 | 608 | 601 |
| 25 | 701 | 685 | 685 | 695 |

TABLE 7
Skin Stripping Study, % Change in Erythema
Adjusted For Normal Skin (410)

| | Zimycan | Zimycan | % | Veh | Veh | % |
|---|---|---|---|---|---|---|
| | Before | After | Change | Before | After | Change |
| 1 | 123 | 62 | −49.6% | 124 | 108 | −12.9% |
| 2 | 186 | 134 | −28.0% | 196 | 187 | −4.6% |
| 3 | 159 | 124 | −22.0% | 166 | 166 | 0.0% |
| 4 | 166 | 143 | −13.9% | 191 | 186 | −2.6% |
| 5 | 178 | 153 | −14.0% | 151 | 160 | 6.0% |
| 6 | 234 | 167 | −28.6% | 207 | 199 | −3.9% |
| 7 | 218 | 203 | −6.9% | 229 | 234 | 2.2% |
| 8 | 153 | 133 | −13.1% | 163 | 156 | −4.3% |
| 9 | 203 | 196 | −3.4% | 232 | 231 | −0.4% |
| 10 | 175 | 172 | −1.7% | 186 | 185 | −0.5% |
| 11 | 157 | 132 | −15.9% | 149 | 147 | −1.3% |
| 12 | 273 | 233 | −14.7% | 276 | 272 | −1.4% |
| 13 | 196 | 182 | −7.1% | 201 | 201 | 0.0% |
| 14 | 245 | 202 | −17.6% | 248 | 247 | −0.4% |
| 15 | 222 | 198 | −10.8% | 201 | 204 | 1.5% |
| 16 | 165 | 139 | −15.8% | 147 | 133 | −9.5% |
| 17 | 119 | 111 | −6.7% | 144 | 133 | −7.6% |
| 18 | 235 | 187 | −20.4% | 235 | 215 | −8.5% |
| 19 | 139 | 132 | −5.0% | 135 | 127 | −5.9% |
| 20 | 215 | 184 | −14.4% | 232 | 228 | −1.7% |
| 21 | 209 | 199 | −4.8% | 248 | 256 | 3.2% |
| 22 | 264 | 245 | −7.2% | 272 | 263 | −3.3% |
| 23 | 201 | 189 | −6.0% | 195 | 199 | 2.1% |
| 24 | 188 | 151 | −19.7% | 198 | 191 | −3.5% |
| 25 | 291 | 275 | −5.5% | 275 | 285 | 3.6% |
| sum | 4914 | 4246 | −13.6% | 5001 | 4913 | −1.8% |
| mean | 196.56 | 169.84 | −13.6% | 200.04 | 196.52 | −1.8% |

The 0.25% miconazole product was deemed well tolerated, since none of the patients reported any side effects. From these data, it is shown that the miconazole nitrate formulations of the present invention have an anti-inflammatory effect that is separate and apart from its previously known anti-microbial activity.

All publications cited in the specification are indicative of the level of skill of those skilled in the art to which this invention pertains. All these publications are herein incorporated by reference to the same extent as if each individual publication were specifically and individually indicated to be incorporated by reference.

Although the invention herein has been described with reference to particular embodiments, it is to be understood that these embodiments are merely illustrative of the principles and applications of the present invention. It is therefore to be understood that numerous modifications may be made to the illustrative embodiments and that other arrangements may be devised without departing from the spirit and scope of the present invention as defined by the appended claims.

### INDUSTRIAL APPLICABILITY

The imidazole topical formulations of the invention can be employed as therapeutic, dermatological, pharmaceutical, medical and/or cosmetic compositions for the relief of inflammation caused by or otherwise associated with non-microbial skin conditions.

1. A method for treating a non-microbial inflammatory skin condition, comprising administering a topical formulation comprising at least one imidazole or a dermatologically acceptable salt thereof, and a determatologically acceptable carrier to an individual in need thereof.

2. The method of claim 1 wherein the imidazole is selected from the group consisting of miconazole, ketoconazole, econazole and isoconazole.

3. The method of claim 1, wherein the imidazole is miconazole nitrate.

4. The method of claim 1 wherein the imidazole is present in said formulation in an amount of about 0.1% to about 10.0% based on total weight of said formulation.

5. The method of claim 4 wherein the amount of the imidazole is about 0.25% to about 2.0%.

6. The method of claim 1, wherein the inflammatory skin condition is selected from the group consisting of dry skin, severe dry skin, dermatitis, psoriasis, eczema, xerosis, terosis, ichthyosis, epidermolytic hyperkeratosis, infantile seborrhoeic dermatitis, atopic dermatitis, chronic dermatitis, keratoses, pruritis, cradle cap, scales, fresh stretch marks, dermatoses, burns and erythema.

7. The method of claim 1, wherein the inflammatory skin condition is dermatitis.

8. The method of claim 1, wherein the inflammatory skin condition is ichthyosis.

9. The method of claim 1, wherein the inflammatory skin condition is eczema.

10. The method of claim 1, wherein the inflammatory skin condition is psoriasis.

11. The method of claim 1, wherein the carrier is petrolatum.

12. The method of claim 1, wherein the carrier is present in said formulation in an amount of about 90% to about 99.9% based on total weight of said formulation.

13. A method for treating a non-microbial inflammatory skin condition, comprising administering a topical formulation comprising a zinc compound, at least one imidazole or a dermatologically acceptable salt thereof, and a determatologically acceptable carrier to an individual in need thereof.

14. The method of claim 13 wherein the imidazole is selected from the group consisting of miconazole, ketoconazole, econazole and isoconazole.

15. The method of claim 13, wherein the imidazole is miconazole nitrate.

16. The method of claim 13 wherein the imidazole is present in said formulation in an amount of about 0.1% to about 10.0% based on total weight of said formulation.

17. The method of claim 16 wherein the amount of the imidazole is about 0.25% to about 2.0%.

18. The method of claim 13, wherein the inflammatory skin condition is selected from the group consisting of dry skin, severe dry skin, dermatitis, psoriasis, eczema, xerosis, terosis, ichthyosis, epidermolytic hyperkeratosis, infantile seborrhoeic dermatitis, atopic dermatitis, chronic dermatitis, keratoses, pruritis, cradle cap, scales, fresh stretch marks, dermatoses, burns and erythema.

19. The method of claim 13, wherein the inflammatory skin condition is dermatitis.

20. The method of claim 13, wherein the inflammatory skin condition is ichthyosis.

21. The method of claim 13, wherein the inflammatory skin condition is eczema.

22. The method of claim 13, wherein the inflammatory skin condition is psoriasis.

23. The method of claim 13, wherein the carrier is petrolatum.

24. The method of claim 13, wherein the carrier is present in said formulation in an amount of about 60% to about 99.65% based on total weight of said formulation.

25. The composition of claim 13, wherein the dermatologically absorbable zinc compound is selected from the group consisting of: zinc acetate, zinc bacitracin, zinc bromide, zinc caprylate, zinc chloride, zinc citrate, zinc fluoride, zinc formate, zinc glycinate, zinc iodate, zinc lactate, zinc nitrate, zinc nitrite, zinc oleate, zinc oxalate, zinc oxide, zinc permanganate, zinc peroxide, zinc phenolsulfonate, zinc phosphate, zinc propionate, zinc pyrophosphate, zinc ricinoleate, zinc salicylate, zinc selenate, zinc silicate, zinc selenide, zinc sulfate, zinc stearate, zinc sulfide, zinc tannate, zinc tartrate, zinc valerate, zinc peptides, and zinc protein complexes.

26. The method of claim 13, wherein said zinc compound is present in said formulation in an amount of about 0.25% to about 30% based on total weight of said formulation.

27. A method for treating a non-microbial inflammatory skin condition, comprising administering a topical formulation comprising zinc oxide, miconazole or a dermatologically acceptable salt thereof, and petrolatum to an individual in need thereof.

28. A method of enhancing the transdermal penetration of a transdermally administrable chemical comprising administering to the skin at least one transdermally administrable chemical and a transdermal penetration enhancing amount of at least one imidazole.
